(19) United States
(12) Patent Application Publication
(10) Pub. No.: US 2017/0156289 A1
(43) Pub. Date: Jun. 8, 2017

(54) Gait Helper

(71) Applicants: Woojo Chung, Seoul (KR); Youngwoong Yoon, Daegu (KR); Chang Sik Kim, Sungnam (KR)

(72) Inventors: Woojo Chung, Seoul (KR); Youngwoong Yoon, Daegu (KR); Chang Sik Kim, Sungnam (KR)

(21) Appl. No.: 14/958,948

(22) Filed: Dec. 4, 2015

(51) Int. Cl. A01K 13/00; A61L 31/04; A61L 31/16; A61L 31/00; A61D 7/00; A61L 31/08

(52) U.S. Cl. CPC A01K 13/003; A61D 7/00; A01K 13/006; A61L 31/08; A61L 31/16; A61L 31/005; A61L 31/047; A61L 2300/216; A61L 2300/30; A61L 2300/252

(57) ABSTRACT

A gait helper includes an hallow tubular member that is designed to elastically fit on the toenails of an animal to treat or prevent a medical condition such as, for example, lupoid onychodystrophy, moist eczema, arthritis or other joint problems.

### FIELD OF THE INVENTION

The present invention relates generally to a device designed to fit on dog's nail providing various medical benefits such as supplying nutrition substances to the nail, allowing space between toes to maintain dryness between toes, and helping gait of the dog on hard or slippery surface floor.

### BACKGROUND OF THE INVENTION

There are various medical conditions relating to dog's toenails or paws. For example, dry, brittle nails are most commonly due to a skin disease known as lupoid onychodystrophy. Certain breeds are more easily affected than same other breeds. The disease causes the immune system to become overactive, attacking the nails and nail beds, creating fissured or split nails that never completely heal. Treatment is challenging, and can take as long as six months to see positive results, lupoid onychodystrophy is treated with a variety of medications, including vitamin E, fish oil (omega fatty acids) and a combination of medications.

Another condition called, “moist eczema” is caused by various reasons such as allergic reactions, scratching or biting, infection, or wetting. While treating the source of the problem would be the primary care, maintaining affected areas dry is also important. When such a problem exists between toenails, keeping the area between the affect toenails is not easy.

Because the nail problems are so painful and uncomfortable, such problems interfere with dog's normal movement. It may be desired to provide additional protection and support on the affected nails helping the movement. Also, these is a need of a mean providing relevant medications directly on the nails over time while the medications is protected from the dog's licking.

Certain nail problems as well as stressful movements by dogs could cause arthritis or other joint problems. Providing adequate supports to dogs could reduce a risk of developing such conditions.

Accordingly a need exists for a device solving the above problems.

### SUMMARY OF THE INVENTION

In one embodiment, the present invention provides a device that may be used to provide medical substances directly on dog's toenails while providing protection on the toenails and addition support on toenail. For example, a tubular gait helper having a hallow tubular member having an internal surface, an outer surface, a proximal end and a distal end, wherein the hallow tubular member has a portion at or near the distal end, which is thicker than the thickness of the hallow tubular member at the proximal end can be used. The hallow tubular member is adopted to elastically fit on a nail of a dog so that said hallow tubular member remains on the nail without any holding devices while the dog is walking.

In another embodiment the present invention provides a tubular gait helper including a hallow tubular member having an internal surface, an outer surface, an outer diameter, an internal diameter, a length, a proximal end and a distal end, wherein said hallow tubular member is adopted to elastically fit on a nail of a dog so that said hallow tubular member remains on the nail without any holding devices while the dog is walking, wherein the outer diameter is between about 4 mm and 9 mm, the internal diameter is between about 2 mm and 4 mm, and the length is between about 4 mm and 8 mm and wherein the ratio between the internal diameter and the outer diameter is between about 1.9 and 2.5 and the ratio between the internal and the length is between about 1.5 and 2.0.

In another embodiment, the present invention provides a tubular gait helper including a hallow tubular member having an internal surface, an outer surface, a thickness, an internal diameter, a proximal end and a distal end, wherein the hallow tubular member is adopted to elastically fit on a nail of a dog so that the hallow tubular member remains on the nail without any holding devices while the dog is walking and wherein at least a portion of the outer surface has a plurality of grooves.

Any of the embodiments described can have a layer containing one or more medical or nutrition substances, for example, selected from glycerin, sweet almond oil vitamin A, vitamin E, cod liver oil and hydrolyzed keratin, on the internal surface where the nail makes a direct contact. Other medical or nutrition substances can also be used in the present invention. The internal surface may have one or more reservoirs capable of holding the medical or nutrition substances. With the reservoirs, more medical or nutrition substances can be held.

Two of the gait helpers can be joined together using a bridge, where the bridge is made of an elastic material. The bridge provides space between two toenails helping on maintaining dry conditions between the toes. The bridge may be elastically expandable allowing movements of the toes but may maintain a minimum length for the spacing effect.

### BRIEF DESCRIPTION OF THE DRAWINGS

FIG. 1 is a perspective view of the embodiment in accordance with the present invention.

FIG. 2 is a side view of the embodiment shown in FIG. 1.

FIG. 3 is a bottom view of the embodiment shown in FIG. 1.

FIG. 4 is a perspective view of another embodiment in accordance with the present invention.

FIG. 5 is a bottom view of the embodiment shown in FIG. 4.

FIG. 6 is a perspective view of another embodiment in accordance with the present invention.

FIG. 7 is a side view of the embodiment shown in FIG. 6.

FIG. 8 is a perspective view of another embodiment in accordance with the present invention.

FIG. 9 is a perspective view of another embodiment in accordance with the present invention.

FIG. 10 is a perspective view of another embodiment in accordance with the present invention.

FIG. 11 is a perspective view of another embodiment in accordance with the present invention.

FIG. 12 is a perspective view of another embodiment is accordance with the present invention.

FIG. 13 is an example illustration showing the use of an embodiment in accordance with the present invention

FIG. 14 is a perspective view of another embodiment in accordance with the present invention

### DETAILED DESCRIPTION OF EXAMPLE EMBODIMENTS

The present invention relates to a device that elastically fit on the toenails of an animal to treat a medical condition such as, for example, lupoid onychodystrophy, moist eczema, arthritis or other joint problems, etc.

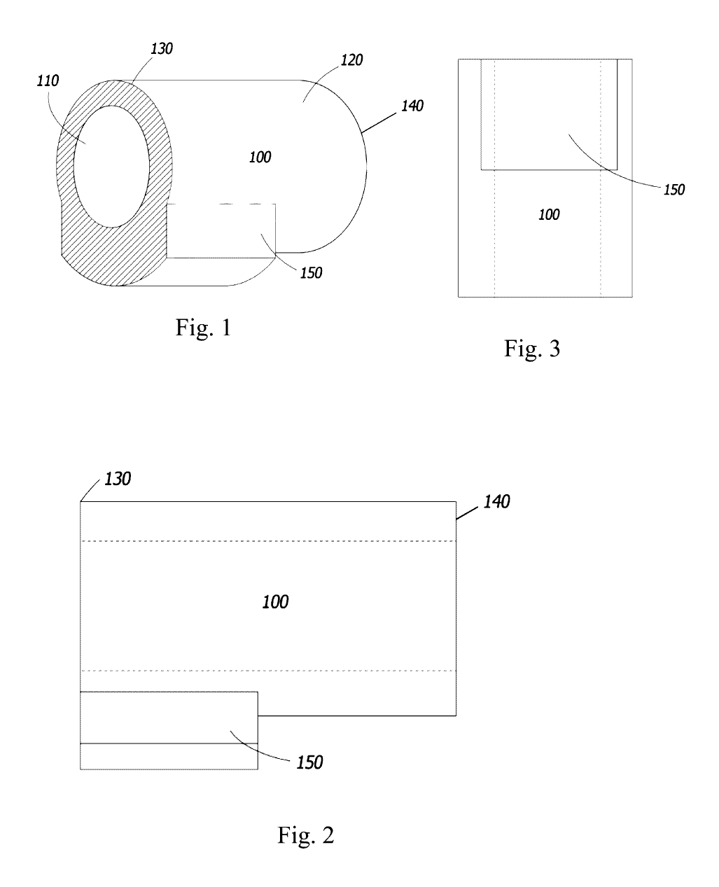

FIG. 1 shows an exemplary embodiment in accordance with the present invention. The hallow tubular member 100 has an internal surface 110, an outer surface 120, a proximal end 130 and a distal end 140, wherein the hallow tubular member has a portion 150 at or near the distal end, which is thicker than the thickness of the hallow tubular member at the proximal end. The hallow tubular member 100 is adopted to elastically fit on a nail of a dog so that the hallow tubular member remains on the nail without any holding devices while the dog is walking. When the hallow tubular member is placed on a nail of a dog, the thicker portion is placed toward the tip of the nail so that the thick portion makes direct contact with the floor when the dog is step on. The hallow tubular member is made of an elastic material and provide additional friction when the dog steps on a hard or slippery floor.

The thicker portion may exist around the entire perimeter of the proximal end or at only a portion of the perimeter. While the thickness of the thicker portion may be made constant throughout its length, it may be made gradually thicker as it toward to the distal end allowing additional surface contact with the floor when the dog steps on it.

The hallow tubular member fits on a toenail elastically and holds the toenail tightly together. It protects the toenail and relieve pain when the dog moves on hard or slippery floors.

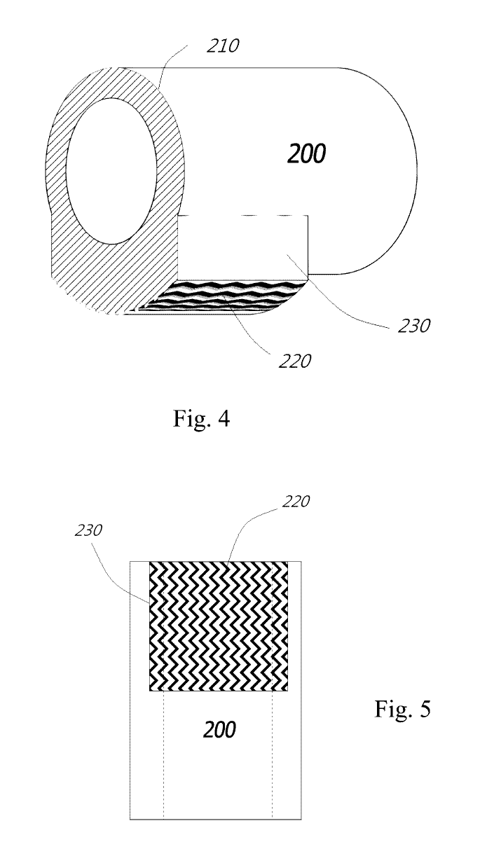

As shown in FIGS. 4, 5 and 8, a plurality of groove may be made on the bottom surface of the thicker portion in order to provide uneven surface, which can allow additional traction. Different shapes of groove can be adopted. The direction of the groove shown in the exemplary embodiments shown in the figures is parallel with the hallow tubular member direction. However, the groove direction can be changed for optimal traction. For example, a groove may be formed in the direction parallel to the peripheral of the proximal end, effectively forming double layers of the thicker portion. The groove may adopt structures to provide additional traction. For example, different shapes used in Tire industry can be adopted.

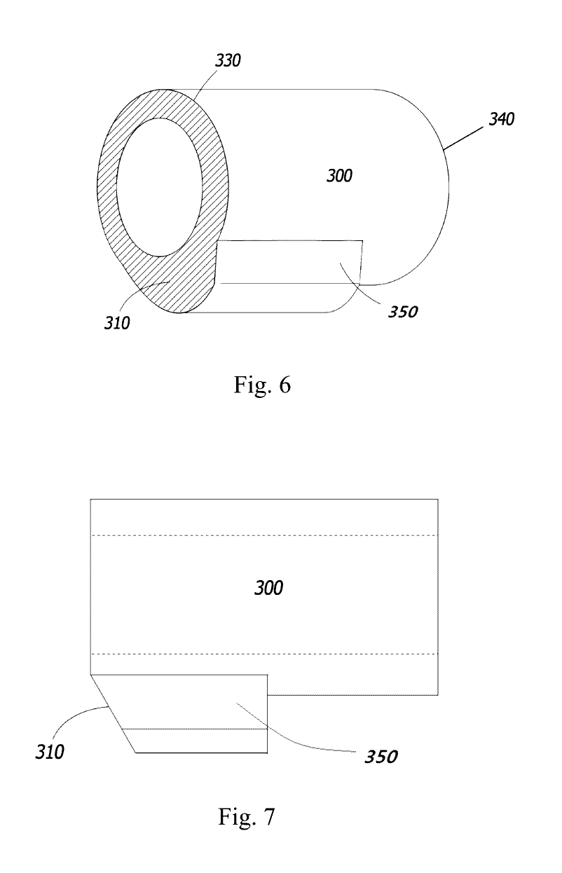

Another embodiment is shown in FIGS. 6 and 7. The hallow tubular member 300 has a thicker portion 350 toward the proximal end 330. The thicker portion 350 has an angled surface 310, which can be 35-55 degree slanted, preferably 45 degree. The angled surface can be flat or contoured. When the angled surface is contoured, the slanted degree is measured by an artificial line connecting points where the contour begins and ends. A plurality of groove may be made on the angled surface and/or the bottom surface of the thicker portion.

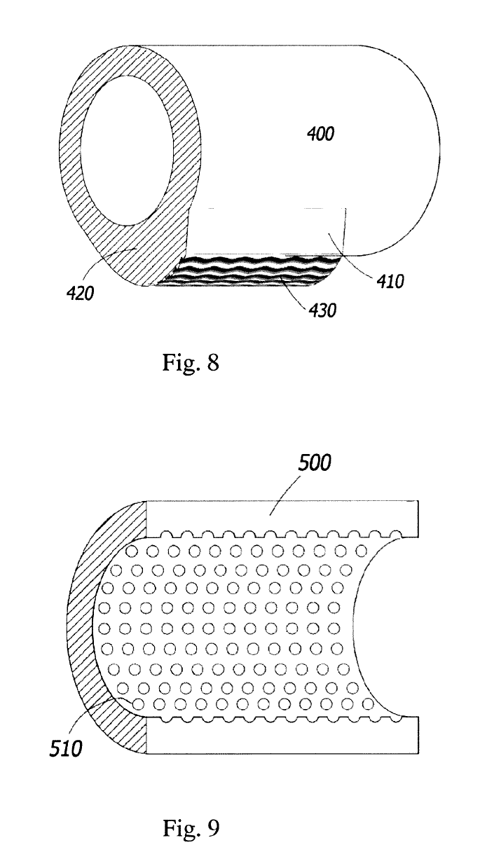

The embodiment 400 shown in FIG. 8 has a thicker portion 410 with an angled portion 426 and the bottom surface of the thicker portion 410 has a plurality of groove 430 providing addition traction.

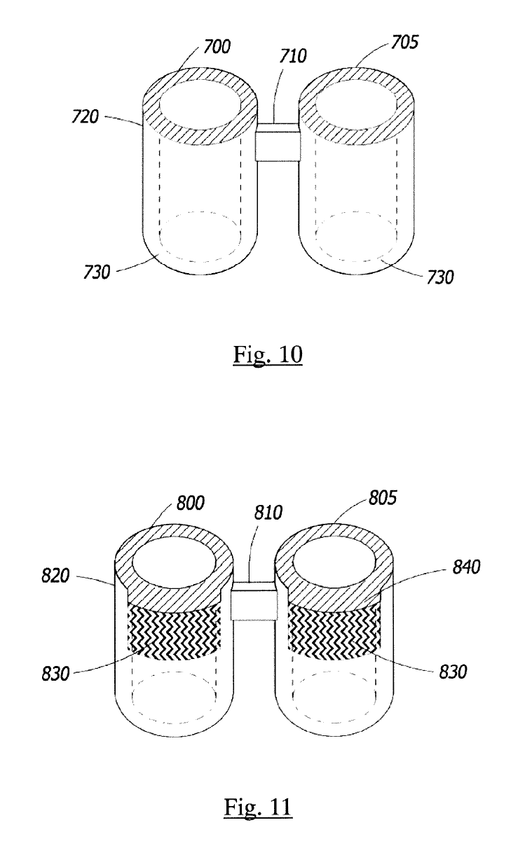

The hallow tubular member described herein may have one or more reservoirs on its internal surface as shown in FIG. 9. The reservoirs can hold one or more medicine substance or nutrition substance such as but not limited to glycerin, sweet almond oil, vitamin A, vitamin E, cod liver oil and hydrolyzed keratin, preferably a mixture of glycerin, sweet almond oil, vitamin E and hydrolyzed keratin. The size of the reservoirs can determined based on the viscosity of the substance or the mixture maintained in the reservoirs. For example, if the substance or mixture is highly viscos, the size can be made larger. The substance or the mixture may be applied directly to the internal surface of the hallow tubular member, which does not have any reservoirs. The nutrition or medicine substance is slowly released to the toenail and the hallow tubular member provides an effective means to apply such substance on the toenail over time.

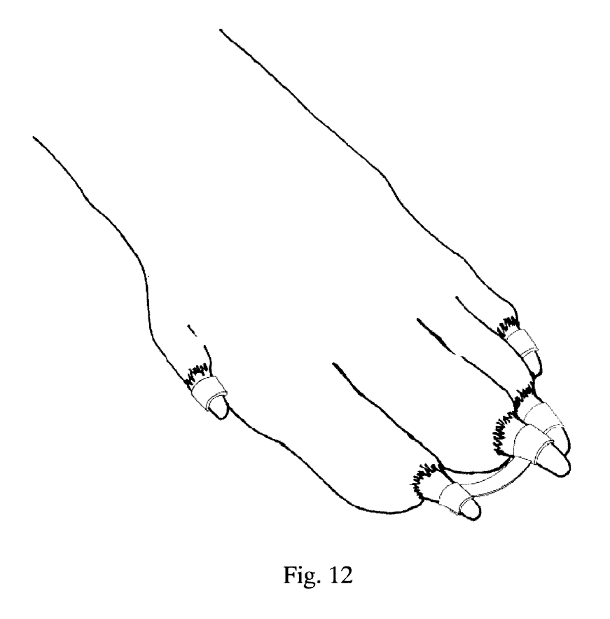

Two of the hallow tubular members can be joined together with a spacer. The spacer is made of an elastic material but should maintain a minimum length to allow space between the two toes. FIGS. 10-12 depict example embodiments incorporating the spacer. The tubular members 700, 705 and 800, 805 are respectively connected to each other by spacers 710, 810. The spacers are located in near the proximal ends 720, 820. The spacer may be made as detachable utilizing convention detachable technologies. The spacer size can be determined based on the size of the dog and is at minimum preferably 4 mm, more preferably 7 mm, even more preferably 10 mm. Since the spacer is made of an elastic material, its length can be extended, which allows movements of the toes outward but maintain the minimum space obtained by the spacer. The embodiment shown in FIG. 12 has a thicker portion 830 near the proximal end 820. The thicker portion has an angled area 840 which can be 35-55 degree slanted, preferably 45 degree. The angled surface can be flat or contoured. The thicker portion may have a plurality of groove providing additional traction.

FIG. 12 illustrates how the embodiments disclosed here are used in the toenails. The one with a spacer is used in the second and third toenails while the other toenails are with single gait helpers in this illustration. The spacer allows maintaining space between the second and third toenails and the dog can help a dry condition between the toenails, which can help treating moist eczema.

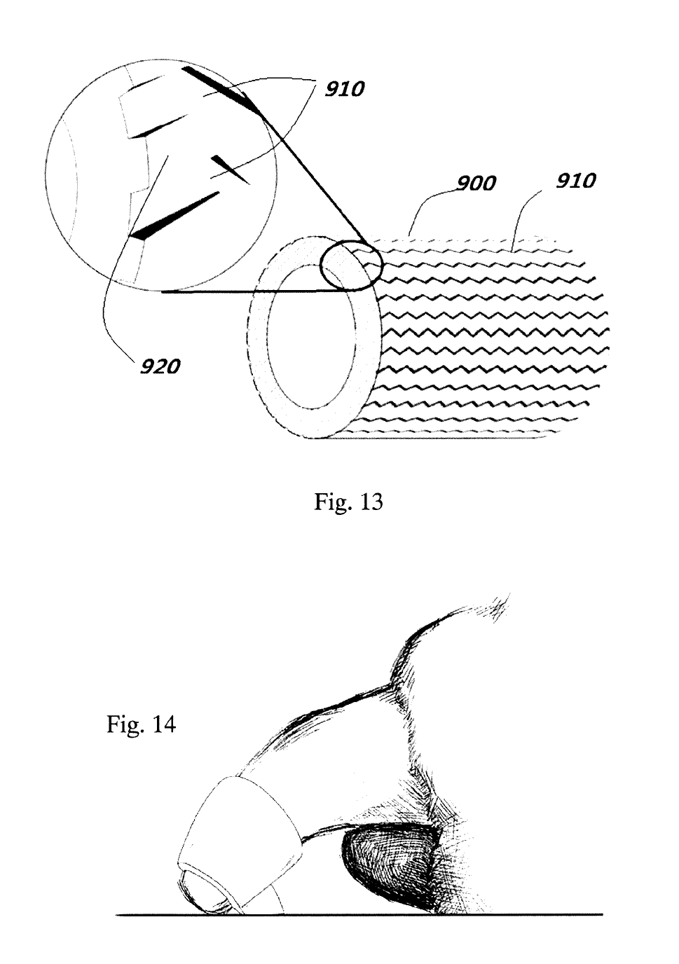

FIG. 13 shows another embodiment where the hallow tubular member 900 has a plurality of protrusion structures 910 creating multiple grooves 920 on the outer surface of the hallow tubular member. Having the grooves all around the outer surface of the hallow tubular member make it easier to apply the tubular member on the toenail because an user does not have to orient the tubular member in a particular direction. Also, while the dog is wearing the tubular member, it may rotate if the tubular member is not tightly placed. This embodiment can work even if such rotation occurs.

FIG. 14 illustrates how an embodiment described here can provide addition traction to the dog. The hallow tubular member is placed on a dog's toenail in a way that the thicker portion faces the floor. The thicker portion makes a direct contact with the floor. Since the thicker portion is made of an elastic material such as rubber, the dog can have a better grip on the floor. With an angled area of the thicker portion, the bottom surface of the thicker portion can easily make a contact with the floor. It can also be designed the angled area is the main contact area rather than the bottom surface of the thicker portion depending on the overall thickness and a designer choice.

The size of the hallow tubular member will depend on the size of the dog to which the hallow tubular member is applied. The internal diameter of the hallow tubular member may be ranged from about 2 to 4 mm. Since the hallow tubular member is made of an elastic material and the hallow tubular member fits to the toenail elastically without any securing device, the internal diameter should be slightly smaller than the toenail's diameter. It is found that the outer diameter is preferably about 4-10 mm, more preferably 5-8. When the hallow tubular member has a thicker portion at or near its proximal end, an optimal thickness may be measured at the thicker portion even if the thicker portion does not cover the entire peripheral of the proximal end. It is found that there are preferred relationship between the internal diameter and the outer diameter. The ration between the former and the latter is preferably about 1:1.4 to 1:2.5, more preferably about 1.5-2.5. It is also found that the ratio between the internal diameter and the length of the hallow tubular member is preferably 1.5-2.4, more preferably 1.7-2.2.

Since the above embodiments are described only for examples, the present invention is not limited to the above embodiments and various modifications or alterations can be easily made therefore by those skilled in the art without departing from the scope of the present invention.

1. A tubular gait helper comprising a hallow tubular member having an internal surface, an outer surface, a proximal end and a distal end, wherein the hallow tubular member has a portion at or near the distal end, which is thicker than the thickness of the hallow tubular member at the proximal end, wherein said hallow tubular member is adopted to elastically fit on a nail of a dog so that said hallow tubular member remains on the nail without any holding devices while the dog is walking.

2. The tubular gait helper according to claim 1, said thicker portion of said hallow tubular member covers less than 50% of the perimeter of said hallow tubular member.

3. The tubular gait helper according to claim 1, said thicker portion of said hallow tubular member has a shoe surface, wherein the shoe surface has one or more grooves providing uneven surface.

4. The tubular gait helper according to claim 1, said internal surface has one or more reservoirs capable of holding one or drugs to be delivered to said nail.

5. The tubular gait helper according to claim 1, further comprising a second hallow tubular member adopted to elastically fit on a nail of a dog, wherein said hallow tubular member and said second hallow tubular member are connected by a bridge providing a predetermined distance between the two hallow tubular member.

6. The tubular gait helper according to claim 5, the bridge is made of an elastic material and capable of maintain a minimum length.

7. The tubular gait helper according to claim 5, the bridge is detachable.

8. The tubular gait helper according to claim 1, wherein the thicker portion has an angled portion toward the distal end.

9. The tubular gait helper according to claim 8, wherein the angled portion has an angle between about 55-35 degrees.

10. The tubular gait helper according to claim 8, wherein the angle is approximately 45 degrees.

11. The tubular gait helper according to claim 1, where the hallow tubular member has an outer diameter, an internal diameter and a length wherein the outer diameter is between about 4 mm and 9 mm, the internal diameter is between about 2 mm and 4 mm, and the length is between about 4 mm and 8 mm and wherein the ratio between the internal diameter and the outer diameter is between about 1.5 and 2.5 and the ration between the internal diameter and the length is between about 1.7 and 2.2.

12. The tubular gait helper according to claim 1, wherein the internal surface has a layer made by applying a solution of glycerin, sweet almond oil, vitamin E and hydrolyzed keratin.

13. A tubular gait helper comprising a hallow tubular member having an internal surface, an outer surface, a thickness, an internal diameter, a proximal end and a distal end, wherein said hallow tubular member is adopted to elastically fit on a nail of a dog so that said hallow tubular member remains on the nail without any holding devices while the dog is walking and wherein at least a portion of the outer surface has a plurality of grooves.

14. (canceled)

15. The tubular gait helper according claim 14, wherein the internal surface has one or more reservoirs capable of holding one or drugs to be delivered to said nail.

16. A tubular gait helper comprising a hallow tubular member having an internal surface, an outer surface, an enter diameter, an internal diameter, a length, a proximal end and a distal end, wherein said hallow tubular member is adopted to elastically fit on a nail of a dog so that said hallow tubular member remains on the nail without any holding devices while the dog is walking, wherein the outer diameter is between about 4 mm and 9 mm, the internal diameter is between about 2 mm and 4 mm, and the length is between about 4 mm and 8 mm and wherein the ratio between the internal diameter and the outer diameter is between about 1.9 and 2.5 and the ration between the internal diameter and the length is between about 1.5 and 2.0.

17. The tubular gait helper according to claim 15, wherein the internal surface contains a layer comprising one or more drug substances selected from glycerin, sweet almond oil, vitamin A, vitamin E, cod liver oil and hydrolyzed keratin.

18. The tubular gait helper according to claim 15, wherein the layer on the internal surface is made by applying a solution of glycerin, sweet almond oil, vitamin E and hydrolyzed keratin.
